(19) United States
(12) Patent Application Publication
(10) Pub. No.: US 2022/0265670 A1
(43) Pub. Date: Aug. 25, 2022

(54) COMPOSITION AND USE THEREOF FOR THE TREATMENT OF CUTANEOUS MASTOCYTOSIS

(71) Applicant: AC BIOSCIENCE SA, Epalinges (CH)

(72) Inventor: Christian AUCLAIR, Saint-Arnoult en Yvelines (FR)

(21) Appl. No.: 17/612,519

(22) PCT Filed: May 22, 2020
(86) PCT No.: PCT/EP2020/064248

§ 371 Date: Nov. 18, 2021

(30) Foreign Application Priority Data
May 22, 2019 (EP) 19176025.5

(51) Int. Cl. A61K 31/5377; A61K 31/352; A61K 31/496; A61K 31/422; A61K 31/427; A61K 31/045; A61K 9/00; A61P 17/02

(52) U.S. Cl. CPC A61K 31/5377; A61K 31/352; A61K 31/496; A61K 31/422; A61K 31/427; A61K 31/045; A61K 9/0014; A61P 17/02

(57) ABSTRACT

The invention provides topical pharmaceutical compositions and use thereof for treating or lessening the symptoms of, or preventing the symptoms of cutaneous mastocytosis.

### FIELD OF THE INVENTION

The invention provides topical pharmaceutical composition and use thereof for treating or lessening the symptoms of, or preventing the symptoms of cutaneous mastocytosis.

### BACKGROUND OF THE INVENTION

Cutaneous mastocytosis describes a group of disorders characterized by the presence of excessive numbers of mast cells in the skin. Patients with cutaneous mastocytosis do not fulfill diagnostic criteria for systemic mastocytosis and show no evidence of organ involvement other than the skin.

Forms of cutaneous mastocytosis include the following three variants: (1) Maculopapular cutaneous mastocytosis (MPCM) or urticaria pigmentosa (UP) with two variants: monomorphic and polymorphic; (2) Diffuse cutaneous mastocytosis and (3) Solitary cutaneous mastocytoma.

Maculopapular cutaneous mastocytosis is also called urticaria pigmentosa. It is the most common type of cutaneous mastocytosis, a condition where there are brown patches or freckles on the skin due to abnormal collections of mast cells.

Diffuse cutaneous mastocytosis (DCM) accounts for around 1-2% of all cases of cutaneous mastocytosis (CM) and almost exclusively presents during infancy, mainly in the neonatal period. Less than 30 cases of neonatal onset DCM have been described in the literature so far. The majority of patients present with generalized erythroderma with a reddish to brown-orange discoloration and extensive bullae. The blisters may become hemorrhagic, may be grouped or linear, and are usually located on the trunk, extremities or scalp. The bullous lesions typically resolve by 3-5 years of age. A small number of patients have been reported with yellow-orange infiltrated and xanthogranuloma-like abnormalities as the presenting feature of DCM (Pseudoxanthomatous DCM). Over time, the skin becomes thickened and has a doughy consistency. Other cutaneous manifestations may include pruritus, urticaria, a positive Darier's sign and marked dermographism. Systemic symptoms (including flushing, hypotension, severe anaphylaxis, hepatomegaly, diarrhea and gastrointestinal bleeding) appear to be more common in DCM than in other forms of CM with systemic symptoms. DCM generally occurs sporadically but a few familial cases have been reported. Mutations in the KIT gene (4q11-q12) have been identified in patients with some forms of mastocytosis and mutations in this gene have been identified in a few patients with DCM. Treatment is symptomatic with administration of antihistamines (H1 and H2 in cases with gastrointestinal symptoms), topical steroids and mast cell membrane stabilizers. Factors that trigger mast cell activation (non-steroidal anti-inflammatory drugs, physical stimuli, emotional stress, insect venom and certain foods) should be avoided. Oral steroid treatment and photochemotherapy with UVA therapy may be of benefit but should only be used in infancy in severe cases that are refractory to alternative treatment options. Close follow-up is required for early detection and management of systemic symptoms.

More than 90% of all patients with mastocytosis initially present with hyperpigmented skin lesions (maculopapular cutaneous mastocytosis [MPCM] or urticaria pigmentosa [UP]). These lesions are disseminated brownish-red macules or slightly elevated papules that may urticate spontaneously or after trauma. This reaction elicited in lesioned skin after stroking or rubbing is referred to as Darier's sign. In children, the lesions tend to be well demarcated, whereas in adults they become confluent and may form raised nodules or plaques. Although lesions may involve all sites of the integument, including mucous membranes, the trunk and proximal extremities typically have the highest density of lesions.

To date, there is no curative treatment of cutaneous mastocytosis and caring is adapted depending on the case, the symptoms, and disease manifestations. Treatments aim either counteracting mediator release symptoms (symptomatic treatments) or reducing organ damage due to neoplastic MC infiltration (non-targeted cytoreductive drugs). KIT-TKIs have recently emerged as promising agents to treat patients with cutaneous mastocytosis.

Therefore, there is still a need for a composition that can efficiently treat cutaneous mastocytosis.

### SUMMARY OF THE INVENTION

An aspect of the present invention provides a pharmaceutical composition comprising therapeutically effective amounts of
Cromolyn and/or one tyrosine kinase inhibitor selected from Masitinib or TKI D816V c-kit,
Terpene alcohol, and
pharmaceutically acceptable excipients and/or carriers.

Further aspect of the present invention provides the pharmaceutical composition of the invention for use in a method for treating or lessening the symptoms of, or preventing the symptoms of cutaneous mastocytosis.

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”.

As used in the specification and claims, the singular form “a”, “an” and “the” include plural references unless the context clearly dictates otherwise.

As used in the specification and claims, the term “and/or” used in a phrase such as “A and/or B” herein is intended to include “A and B”, “A or B”, “A”, and “B”.

As used herein the terms “subject” and “patient” are well-recognized in the art, and, are used herein to refer to a mammal, including, for example, humans, domestic pets, livestock and other farm animals; the most preferably a mammal is a human. In some embodiments, the subject is a subject in need of treatment or a subject having cutaneous mastocytosis. The term does not denote a particular age or sex. Thus, adult, children and newborn subjects, whether male or female, are intended to be covered.

As used herein the term “pharmaceutically acceptable excipients and/or carriers” means that the compositions or components thereof so described are suitable for use in contact with skin of a subject (patient), or suitable for any other means of administration to subject (patient) body without undue toxicity, incompatibility, instability, irritability, allergic response, and the like.

The term “treat” and its grammatical variants (for example “to treat,” “treating,” and “treatment”) refer to administration of an active pharmaceutical ingredient to a subject (patient) with the purpose of ameliorating or reducing the incidence of one or more symptoms of a condition or disease state in the subject (patient). Such symptoms may be chronic or acute; and such amelioration may be partial or complete. In the present context, treatment entails topically administering the pharmaceutical composition of the invention to a subject (patient).

As used herein the term “topical” or “topically” refers to the application of the composition of the present invention onto the surface of the skin and/or a portion thereof.

As used herein the term “administration” or “administering” to human body refers to any means of introducing the composition of the present invention onto and/or into the subject (patient) body or a portion thereof (such as topical administration into and/or onto the skin or portion of the skin or topical application on the skin or portion thereof).

The term “therapeutically effective amount,” as used herein, refers to any amount of a specific component or combination of components that will cause a reduction of symptoms, disappearance of the symptoms or relief from symptoms related to cutaneous mastocytosis, when applied, either once, or repeatedly over time. Therapeutically effective amounts can be readily determined by persons skilled in the art using routine experimentation and using tests and measures commonly employed in the art, or can be based upon the subjective response of patients undergoing treatment.

The term “prophylaxis” and “preventing” refer to administration of an active pharmaceutical ingredient or composition to a subject (patient) with the purpose of reducing the occurrence or recurrence of one or more acute symptoms associated with a disease state or a condition in the subject (patient). In the present context, prophylaxis or preventing entails topically administering the pharmaceutical composition of the invention to a subject (patient). Thus, prophylaxis or preventing includes reduction in the occurrence or recurrence rate of a cutaneous mastocytosis. However, prophylaxis or preventing is not intended to include complete prevention of onset of a disease state or a condition in a subject (patient) who has not previously been identified as suffering from the disease or the condition.

An aspect of the present invention provides a pharmaceutical composition that is used for treating cutaneous mastocytosis. In some embodiments, treatment relates to treating, or lessening the symptoms of, or preventing the symptoms of cutaneous mastocytosis.

According to an embodiment, the pharmaceutical composition of the invention comprises therapeutically effective amounts of
Cromolyn and/or one tyrosine kinase inhibitor selected from Masitinib or TKI D816V c-kit,
Terpene alcohol, preferably Bisabolol, and
pharmaceutically acceptable excipients and/or carriers.

According to another embodiment, the pharmaceutical composition of the invention comprises therapeutically effective amounts of
Cromolyn
Optionally Masitinib or TKI D816V c-kit,
Terpene alcohol, preferably Bisabolol, and
pharmaceutically acceptable excipients and/or carriers.

According to another embodiment, the pharmaceutical composition of the invention comprises therapeutically effective amounts of
Cromolyn,
optionally Masitinib,
Terpene alcohol, preferably Bisabolol, and
pharmaceutically acceptable excipients and/or carriers.

According to another embodiment, the pharmaceutical composition of the invention comprises therapeutically effective amounts of
Cromolyn,
optionally TKI D816V c-kit,
Terpene alcohol, preferably Bisabolol, and
pharmaceutically acceptable excipients and/or carriers.

According to another embodiment, the pharmaceutical composition of the invention comprises therapeutically effective amounts of
A tyrosine kinase inhibitor selected from Masitinib or TKI D816V c-kit,
Terpene alcohol, preferably Bisabolol, and
pharmaceutically acceptable excipients and/or carriers.

According to a further embodiment, the pharmaceutical composition of the invention comprises therapeutically effective amounts of
Cromolyn: 0% to 10%, preferably 0.001% to 10%, preferably 1% to 5%,
Masitinib or TKI D816V c-kit: 0% to 4%, preferably 0.1% to 2%%, preferably 0.5% to 1%
Terpene alcohol, preferably Bisabolol: 05% to 4%, preferably 1% to 2%, preferably 1%
pharmaceutically acceptable excipients and/or carriers,
provided that at least Cromolyn or one tyrosine kinase inhibitor selected from Masitinib or TKI D816V c-kit is present in the pharmaceutical composition.

According to a further embodiment, the pharmaceutical composition of the invention comprises therapeutically effective amounts of
Cromolyn: 2%
TKI D816V c-kit: 0.5%
Bisabolol: 1%
pharmaceutically acceptable excipients and/or carriers.

According to a further embodiment, the pharmaceutical composition of the invention comprises therapeutically effective amounts of
Cromolyn
Terpene alcohol, preferably Bisabolol, and
pharmaceutically acceptable excipients and/or carriers.

According to a further embodiment, the pharmaceutical composition of the invention comprises therapeutically effective amounts of
Cromolyn
Bisabolol, and
pharmaceutically acceptable excipients and/or carriers.

According to a further embodiment, the pharmaceutical composition of the invention comprises therapeutically effective amounts of
Masitinib or TKI D816V
Bisabolol, and
pharmaceutically acceptable excipients and/or carriers.

Cromolyn (or cromoglycate) used in the pharmaceutical compositions of the present invention is selected from the group comprising cromolyn sodium, cromolyn lysinate, and magnesium cromoglycate.

In a preferred embodiment, cromolyn (or cromoglycate) used in the pharmaceutical compositions of the present invention is cromolyn (or cromoglycate) sodium, a mast cell stabilizer with anti-inflammatory activity. Cromolyn sodium probably interferes with the antigen-stimulated calcium transport across the mast cell membrane, thereby inhibiting mast cell release of histamine, leukotrienes, and other substances that cause hypersensitivity reactions. Cromolyn sodium also inhibits eosinophil chemotaxis. Indeed, mast cell degranulation is under the dependence of calcium release and subsequent actin cytoskeleton reorganization. Calcium release through calcineurin NFAT pathway activates the transcription of inflammatory cytokines as well. Calcium sequestration which can be achieved by calcium ionophore results in the inhibition of degranulation and cytokines release. Several compounds have been found to inhibit mast cell activation including chromoglycate (cromolyn) and flavonoids derivatives.

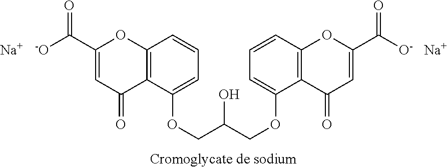

Masitinib used in the pharmaceutical compositions of the present invention is a potent and selective inhibitor of the stem cell factor (SCF) receptor, c-Kit, and the platelet-derived growth factor (PDGFR) receptor kinase. Similar to other tyrosine kinase inhibitors (TKI), masitinib exerts its mode of action via binding to the ATP binding pocket of its target kinases and thereby inhibiting the phosphorylation-dependent signaling pathways. Masitinib shows in vitro activity against the wildtype and mutant c-Kit (exon 9 and exon 11). Furthermore, it also inhibits the PDGF-α and β receptors, Lyn kinase and DDR1 but lacks inhibitory activity against Bcr-Abl and against VEGFR, FGF and FLT3. At the cellular level, masitinib is a selective inhibitor of JM-mutated (exon 11) c-Kit-dependent cell proliferation in the nanomolar range (IC₅₀ 0.005 μM) and WT c-Kit-dependent cell proliferation in the micromolar range (IC₅₀ between 0.1 and Masitinib is able to block PDGF-R-dependent cell proliferation at nanomolar concentrations (IC₅₀ 0.00025-0.02 μM). Its main metabolite, the N-desmethylated derivative AB3280, has essentially the same inhibitory profile as masitinib, even though slightly less potent than masitinib. Furthermore, masitinib inhibits Lyn-mediated phosphorylation in a recombinant enzymatic assay at submicromolar concentrations (IC₅₀: 0.22±0.40 μM). This property explains the significant inhibition of mast degranulation at low masitinib concentrations. Masitinib inhibition is highly selective for c-Kit in comparison to other kinases (including EGFR, RET, TRKB, FGFR1, FGFR3, and FLT3). The knowledge of the selectivity of protein kinase inhibitors (PKIs) is a critical point for the development of optimal safe and well tolerated compounds in human health, particularly for the treatment of non-lethal inflammatory diseases.

The TKI D816V c-kit (c-KIT D816V) inhibitors are tyrosine kinase inhibitors used in the pharmaceutical compositions of the present invention can be selected among compounds displaying optimal balance between inhibitory strength and selectivity.

In preferred embodiment of the present invention, TKI D816V c-kit (c-KIT 816V) inhibitors used in the pharmaceutical compositions of the present invention is a compound of Formula I

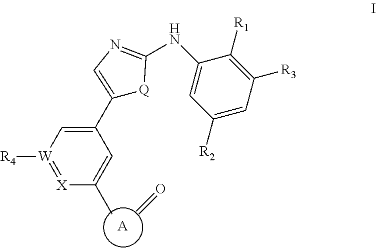

wherein
R₁ is C₁-C₄ alkyl, preferably methyl;
R₂ is selected from the group comprising —CH₂—O—CH₂—CH₃, —CH₂—O—CH₃, —O—CH₃,
O—(CH₂)₂—OH,

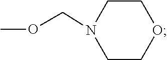

R₃ is hydrogen
R4 is selected from the group comprising

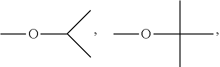

### —O—CF₃;

Q is O or S
W is C
X is C or N
A is selected from the group comprising

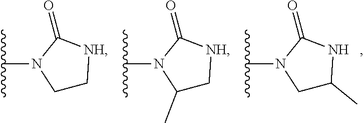

C₅-C₈ heterocycloalkyl containing one to four heteroatoms selected from O, N, and S, preferably 1 or 2 heteroatoms.

In some embodiments of the invention, the compounds of Formula I are selected from the group comprising:

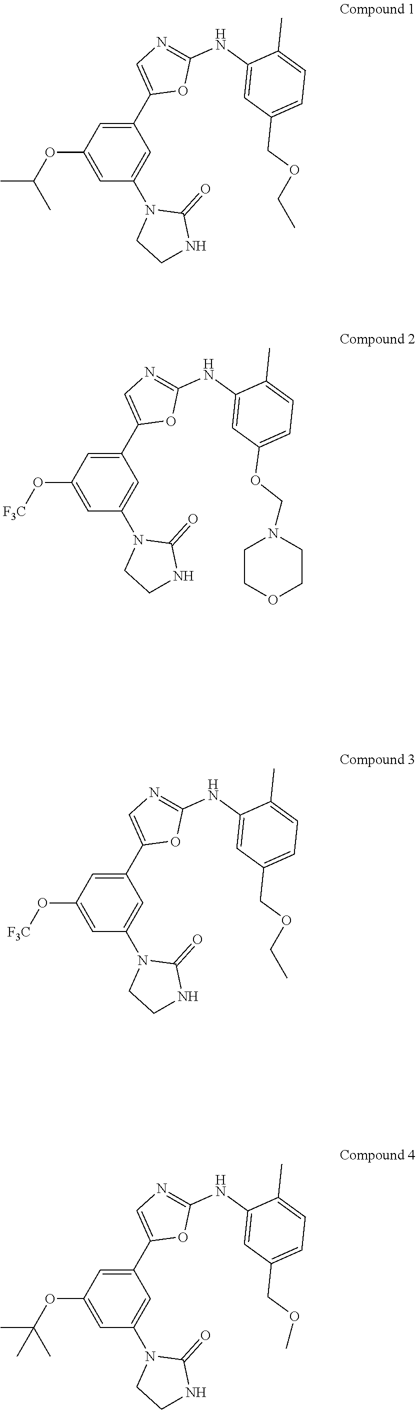 [SMILES: CCOCc1ccc(C)c(Nc2ncc(-c3cc(OC(C)C)cc(N4CCNC4=O)c3)o2)c1 ; Cc1ccc(OCN2CCOCC2)cc1Nc1ncc(-c2cc(OC(F)(F)F)cc(N3CCNC3=O)c2)o1 ; CCOCc1ccc(C)c(Nc2ncc(-c3cc(OC(F)(F)F)cc(N4CCNC4=O)c3)o2)c1 ; COCc1ccc(C)c(Nc2ncc(-c3cc(OC(C)(C)C)cc(N4CCNC4=O)c3)o2)c1]

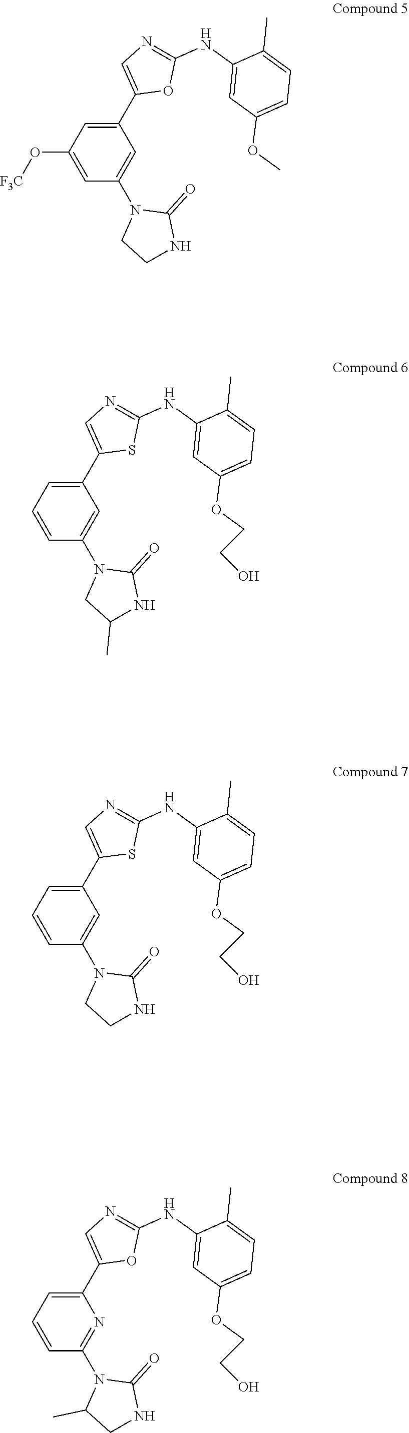 [SMILES: COc1ccc(C)c(Nc2ncc(-c3cc(OC(F)(F)F)cc(N4CCNC4=O)c3)o2)c1 ; Cc1ccc(OCCO)cc1Nc1ncc(-c2cccc(N3CC(C)NC3=O)c2)s1 ; Cc1ccc(OCCO)cc1Nc1ncc(-c2cccc(N3CCNC3=O)c2)s1 ; Cc1ccc(OCCO)cc1Nc1ncc(-c2cccc(N3C(=O)NCC3C)n2)o1]

In other embodiments, TKI D816V c-kit (c-KIT 816V) inhibitors used in the pharmaceutical compositions of the present invention can be selected from chemical group of molecules displaying a significant inhibition on the catalytic activity of c-Kit mutated at the D816V position

In a preferred embodiment of the present invention, the terpene alcohol used in the pharmaceutical compositions of the present invention is selected from the group comprising a monoterpene alcohol, a sesquiterpene alcohol or a diterpene alcohol and combinations of one or more thereof. Preferably, the one or more terpene alcohol is selected from the group comprising cedrenol, cedrols, geraniol, nerolidol, bisabolol, citronellol, nerol, terpineol, linalool, menthol, pulegol, carveol, pinocampheol, myrcenol, isopulegol, farnesol, lanceol, santalols, vetiverol, viridiflorol, valerianol, tumerols, patchoulol, occidol, nootkatol, jinkoh eremol, hanamyol, guaicol germacradienol, fokienol, eudesmols, and cadinols, an active optical or steric isomer of these compounds and combinations of one or more thereof. The most preferably, the terpene alcohol is bisabolol.

Bisabolol (or α-Bisabolol) used in the pharmaceutical compositions of the present invention is a naturally occurring sesquiterpene alcohol which was first isolated from Matricaria chamomilla (Asteraceae) in the twentieth century and has since been identified in other aromatic plants such as Eremanthus erythropappus, Smyrniopsis aucheri and Vanillosmopsis species. α-Bisabolol was identified as a major constituent of Salvia runcinata essential oil, a plant indigenous to South Africa. Alpha-Bisabolol is an inflammatory-inhibiting sesquiterpene (6-methyl-2-(4-methyl-3-cyclohexen-1-yl)-5-hepten-2-ol; 1-methyl-4-(1,5-dimethyl-1-hydroxyhex-4(5)-nyl) cyclohexen-1) used in cosmetics and personal care products and is found in various plants, including the herbal tea, chamomile. The most important known effects of Bisabolol are anti-inflammatory, wound-healing, anti-bacterial, anti-mycotic, anti-phlogistic. In addition, α-Bisabolol was assessed for its ability to enhance transepidermal drug penetration, predominantly arising from an increase in their diffusivities across the skin barrier. Bisabolol can decrease leukocyte migration, protein extravasations and the amount of TNF-α released to the peritoneal cavity in response to carrageenan. In the same way, bisabolol reduces the influx of cells inflammatory (neutrophils) in the gastric mucosa in gastric ulcer induced by absolute ethanol.

The pharmaceutical compositions of the invention comprise also pharmaceutically acceptable excipients and/or carriers for topical use, as are used conventionally in such compositions, for example preservatives, antioxidants, bactericides, fungicides, solvents, perfumes, substances for preventing foaming, dyestuffs, pigments which have a coloring effect, thickeners, propellants, surfactant substances, emulsifiers, softening, moisturizing and/or moisture-retaining substances, distilled water, fats, oils, waxes or other conventional constituents of a topical composition, such as alcohols, polyols, polymers, foam stabilizers, electrolytes, organic solvents or silicone derivatives. The necessary amounts of the pharmaceutically acceptable excipients and/or carriers can, based on the desired product, easily be chosen by a person skilled in the art.

According to an embodiment of the present invention, the pharmaceutical composition of the invention is a cream, water based O/W, comprising the following excipients and/or carriers: water, sweet almond oil, caprylic/capric triglyceride, olus oil, butyrospermum parkII butter, dicaprylyl ether, glycerin, propanediol, alcohol denat, sorbitol, cetearyl alcohol, glyceryl stearate, glyceryl stearate citrate, polyglyceryl-3 methylglucose distearate, xanthan gum, sodium dehydroacetate, sodium benzoate, phenoxyethanol, citric acid.

The pharmaceutical compositions of the invention can take various forms, depending on topical application. For example, the pharmaceutical compositions for topical administration can be in the form of ointments, lotions, creams, foams, gels, solutions patches or sprays. The pharmaceutical compositions can also be incorporated into dedicated applicators, such as saturated pads, to facilitate administration to the skin.

The pharmaceutical compositions of the invention can be packaged to provide a single dose or multiple doses, and to provide a convenient means of transport, handling, and administration. The pharmaceutical compositions can be also packaged in such a way as to protect the composition from oxidation, bacterial contamination, or other forms of deterioration or degradation. For example, the pharmaceutical compositions of the invention for topical administration can be packaged into crimped tubes, airless containers, or sealed foil-lined packets, which may optimally contain enough of the composition for a single application, or a limited number of applications. The pharmaceutical compositions of the invention for topical administration can be packaged in larger containers designed for multiple applications. When packaged in such larger containers, those containers may be equipped with pumps or other mechanisms designed to facilitate the delivery of an appropriate volume of the composition, while reducing the likelihood of contamination or oxidation.

According to an embodiment of the invention, the pharmaceutical composition of the invention is a water-based cream (O/W) comprising the following pharmaceutically acceptable excipients and/or carriers:
AQUA to 100%
SWEET ALMOND OIL 12.5%
CAPRYLIC/CAPRIC TRIGLYCERIDE 5.0%
OLUS OIL 5.0%
BUTYROSPERMUM PARKII BUTTER 3.0%
DICAPRYLYL ETHER 3.0%
GLYCERIN 3.0%
PROPANEDIOL 3.0%
ALCOHOL DENAT 3.0%
SORBITOL 2.1%
CETEARYL ALCOHOL 2.0%
GLYCERYL STEARATE 1.5%
GLYCERYL STEARATE CITRATE 1.5%
POLYGLYCERYL-3 METHYLGLUCOSE DISTEARATE 1.0%
XANTHAN GUM 0.4%
SODIUM DEHYDROACETATE 0.1%
SODIUM BENZOATE 0.3%
PHENOXYETHANOL 0.6%
CITRIC ACID 0.001%

The pharmaceutical compositions of the invention are intended for use on mammalian skin, including, for example, the skin of humans, domestic pets, livestock and other farm animals.

When used on human subjects, or human patients in need of such treatment, the human patients may be of any age or gender, although specific compositions may be developed for treating human patients within specific age ranges, or of a particular gender.

The pharmaceutical compositions of the invention are intended to treat skin lesions (disorders or conditions of the skin), specifically the symptoms, resulting from cutaneous mastocytosis.

The pharmaceutical compositions of the invention can be also be used prophylactically, in order to prevent, protect and/or lessen the symptoms of cutaneous mastocytosis.

Another aspect of the present invention provides the pharmaceutical composition of the invention for use in a method for treating or lessening the symptoms of, or preventing the symptoms of cutaneous mastocytosis. In a specific embodiment, the present invention provides the pharmaceutical composition of the invention for use in a method for treating the symptoms of cutaneous mastocytosis. In another specific embodiment, the present invention provides the pharmaceutical composition of the invention for use in a method for lessening the symptoms of cutaneous mastocytosis. In a further specific embodiment, the present invention provides the pharmaceutical composition of the invention for use in a method for preventing the symptoms of cutaneous mastocytosis.

Skin lesions are characteristic symptoms of cutaneous mastocytosis. The symptoms known to occur in cutaneous mastocytosis are selected from the group comprising small areas of skin that change colour (macules), small firm raised bumps (papules), larger raised red bumps (nodules), large raised areas of skin noticeable to the touch (plaques), blisters that mainly affect young children with mastocytomas (tumours consisting of mast cells) or diffuse cutaneous mastocytosis (a rare form of cutaneous mastocytosis), itchy and brown patches on the skin, itchy and red areas on the skin.

The symptoms of cutaneous mastocytosis are selected from criteria established by the European Union-US consensus group. These criteria include the presence of typical skin lesions, a histological confirmed infiltrate of mast cells in the dermis and in some cases (mainly adults), the presence of an activating c-kit mutation at codon 816 in lesioned skin.

Skin lesions due to cutaneous mastocytosis usually develop on the trunk rather than the head, neck and limbs.

In some embodiments, the method comprises applying the pharmaceutical compositions of the invention to the affected skin of a subject (patient) once a day. In other embodiments the method comprises applying the pharmaceutical compositions of the invention to the affected skin of a subject (patient) multiple times a day.

Further aspect of the present invention provides a method of treating or lessening the symptoms of, or preventing the symptoms of cutaneous mastocytosis, comprising administering the pharmaceutical composition of the invention to the affected skin of a subject (patient).

In some embodiments, the method comprises applying the pharmaceutical compositions to the affected skin of a subject (patient) once a day. In other embodiments the method comprises applying the pharmaceutical compositions to the affected skin of a subject (patient) multiple times a day.

The methods of treatment to be employed with the pharmaceutical compositions of the invention will vary depending upon the disorder, or condition, or symptom to be treated, and its severity. The methods will also vary depending upon the nature of the subject to be treated; their species, gender, and age, etc. Optimal methods of treatment, including the choice of specific formulation, the form of that formulation, the frequency of administration, and the duration of treatment will be adjusted according to the response of the patient, and the efficacy of the treatment, as will be judged by the patient themselves, or by a health care provider who is directing the treatment. Specific details regarding the methods of treatment can be defined by a health care provider overseeing the treatment, or by the patient, as results are obtained. Effective results will, in most cases, be achieved by topical application of the pharmaceutical compositions of the invention in a thin layer directly over the affected area or areas, or in the area where one seeks to obtain a desired result.

Depending upon the skin lesion, disorder, condition, or symptom to be treated, and its severity, and whether the treatment is being done for therapeutic or prophylactic reasons, effective results may be obtained with application rates of from one application every week, to once every day, to multiple applications per day. The duration of the treatment regimen can be adjusted according to the patient's needs and according to the disorder's response to the treatment. Treatment can either be discontinued, or its frequency lessened, once symptoms diminish or disappear. Alternatively, it may be advantageous for treatments to continue for a fixed period beyond the diminution or disappearance of symptoms, and in other cases, it may be advantageous for treatment to continue indefinitely as a prophylactic treatment in patients who suffer from chronic cutaneous mastocytosis.

Cutaneous mastocytosis is usually caused by changes (mutations) in the KIT gene. The target population suffering from cutaneous mastocytosis consists of children and adult population. In children, 70% of population (CA population) have no detectable mutation or c-kit mutations K5091, ITD501-502, ITD502-503, Del 419; and 30% of population (CB population) have c-kit D816V mutation. In adult, 30% of population (AA population) have no detectable mutation and no c-kit D816V mutation and 70% of population (AB population) have c-kit D816V mutation. Indeed, despite the fact that most patients with cutaneous mastocytosis harbor the imatinib-resistant KIT D816V mutant, it is of utmost importance to determine the KIT mutational status in each patient before applying KIT-TKIs, as some patients may present with KIT WT, or KIT mutant outside exon 17, potentially responsive to imatinib.

Indeed, several gain-of-function-mutations in the kinase domain of c-kit appear to occur in mastocytosis supporting the clonal (neoplastic) nature of the disease. Also, certain point mutations appear to be associated with distinct variants of mastocytosis, i.e. Asp-816→Val with a subset of sporadic persistent (systemic) mastocytosis (mostly adults), and GLY-839→Lys with (a subset of) typical pediatric (mostly cutaneous) mastocytosis. Polymerase chain reaction and direct sequencing using genomic DNA samples from 16 nonfamilial Japanese patients with indolent cutaneous mastocytosis (12 with childhood-onset disease and 4 with adult-onset disease) have been performed to look for the most common c-kit mutations at codons 816, 560, 820, and 839 (Yanagihori et al, 2005). A substantial number of patients had missense codon 816 mutations (10 of 12 in the childhood-onset group, 83.3%; and 4 of 4 in the adult-onset group, 100%). The most common mutation was Asp816Val (9 of 16, 64.3%) followed by Asp816Phe (5 of 16, 35.7%). Moreover, children with the Asp816Phe mutation developed cutaneous mastocytosis at an earlier age as compared to those with the Asp816Val mutation (mean age of onset, 1.3 months versus 5.9 months, respectively; P=0.068).

In one embodiment the present invention relates to the use or the method as defined above wherein patients are those afflicted with mastocytosis with mast cell mediator release associated handicap, and in particular cutaneous mastocytosis, wherein said patients have a positive D816V c-Kit mutation status. For this group of patients, the pharmaceutical composition of the invention can also comprise therapeutically effective amount of TKI D816V c-kit. Thus in an embodiment, the pharmaceutical composition of the invention comprises therapeutically effective amounts of:
Cromolyn,
TKI D816V c-kit,
Terpene alcohol, preferably Bisabolol, and
pharmaceutically acceptable excipients and/or carriers.

or in another embodiment, the pharmaceutical composition of the invention comprises therapeutically effective amounts of:
TKI D816V c-kit,
Terpene alcohol, preferably Bisabolol, and
pharmaceutically acceptable excipients and/or carriers.

In another embodiment the present invention relates to the use or the method as defined above wherein patients are those afflicted with mastocytosis with mast cell mediator release associated handicap, and in particular cutaneous mastocytosis, wherein said patients have a negative D816V c-Kit mutation status. For this group of patients, the pharmaceutical composition of the invention can also comprise therapeutically effective amount of masitinib. Thus in an embodiment, the pharmaceutical composition of the invention comprises therapeutically effective amounts of:
Cromolyn,
Masitinib,
Terpene alcohol, preferably Bisabolol, and
pharmaceutically acceptable excipients and/or carriers.

or in another embodiment, the pharmaceutical composition of the invention comprises therapeutically effective amounts of:
Masitinib,
Terpene alcohol, preferably Bisabolol, and
pharmaceutically acceptable excipients and/or carriers.

An aspect of the present invention provides a method for treating or lessening the symptoms of, or preventing the symptoms of cutaneous mastocytosis comprising
determining the status of D816V c-kit mutation in a subject,
administering the pharmaceutical composition of the invention comprising therapeutically effective amounts of:
   optionally Cromolyn,
   Masitinib,
   Terpene alcohol, preferably Bisabolol, and
   Pharmaceutically acceptable excipients and/or carriers.
to a subject having negative D816V c-kit mutation status, or
administering the pharmaceutical composition of the invention comprising therapeutically effective amounts of:
   optionally Cromolyn,
   TKI D816V c-kit,
   Terpene alcohol, preferably Bisabolol, and
   Pharmaceutically acceptable excipients and/or carriers.
to a subject having positive D816V c-kit mutation status.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

The foregoing description will be more fully understood with reference to the following Examples. Such Examples, are, however, exemplary of methods of practising the present invention and are not intended to limit the application and the scope of the invention.

### EXAMPLES

### Example 1: Feasibility of the Topical Formulations

Various formulations have been prepared to evaluate the compatibility and stability of active molecules when dispersed in the oil/water emulsion and to evaluate the physicochemical parameters of the formulation.

The following formulations have been successfully prepared:

Formulation I

Cromoglycate 1%

Bisabolol 1%

Emulsion base

Formulation II

Cromoglycate 5%

Bisabolol 2%

Emulsion base

Formulation III

Cromoglycate 1%

Masitinib 0.5%

Bisabolol 1%

Emulsion base

Formulation IV

Compound c-KIT D816V inhibitor 0.5% (Compound 1, 3 and 5 disclosed above)

Bisabolol 1%

Emulsion base

Formulation V

Compound c-KIT D816V inhibitor 1% (Compound 1, 3 and 5 disclosed above)

Bisabolol 2%

Emulsion base

### Example II: Test of Efficacy

An exploratory test of efficacy has been performed on a patient suffering from indolent mastocytosis with cutaneous lesions and the occurrence of a c-KIT D816V mutation.

Formulation V (Example I) was applied on cutaneous lesions twice a day for one week. It has been observed a marked decrease of the size and intensity of the cutaneous lesions. More surprisingly it has been observed a decrease of systemic symptoms usually resulting from histamine release such as headache and insomnia.

1. A pharmaceutical composition comprising a therapeutically effective amounts of
cromolyn and/or a tyrosine kinase inhibitor selected from Masitinib and TKI D816V c-kit,
terpene alcohol, and
a pharmaceutically acceptable excipient and/or carrier.

2. The pharmaceutical composition of claim 1, wherein the TKI D816V c-kit is a compound of Formula I 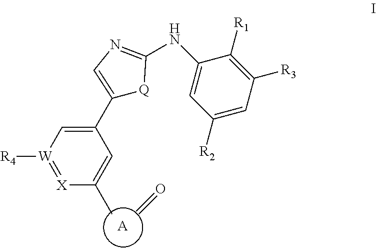
wherein
R₁ is C₁-C₄ alkyl;
R₂ is selected from the group consisting of —CH₂—O—CH₂—CH₃, —CH₂—O—CH₃,
—O—CH₃, —O—(CH₂)₂—OH, 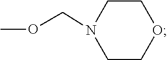
R₃ is hydrogen;
R4 is selected from the group comprising 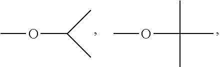
—O—CF₃;
Q is O or S;
W is C;
X is C or N;
A is selected from the group consisting of 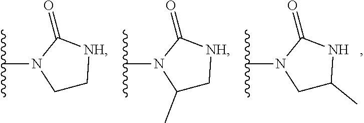
and C₅-C₈ heterocycloalkyl containing one to four heteroatoms selected from O, N, and S.

3. The pharmaceutical composition of claim 1, wherein TKI D816V c-kit is selected from the group consisting of

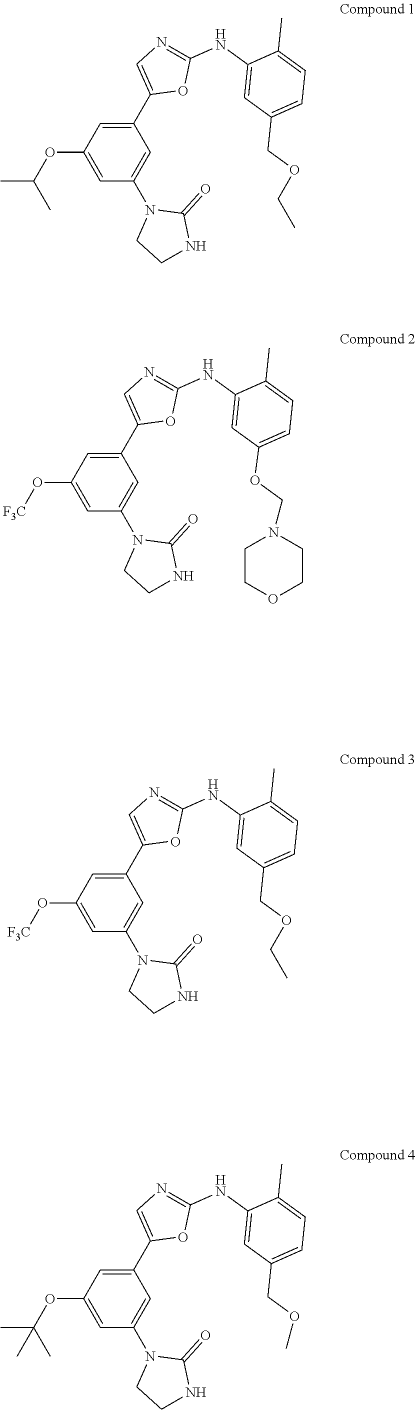 [SMILES: CCOCc1ccc(C)c(Nc2ncc(-c3cc(OC(C)C)cc(N4CCNC4=O)c3)o2)c1 ; Cc1ccc(OCN2CCOCC2)cc1Nc1ncc(-c2cc(OC(F)(F)F)cc(N3CCNC3=O)c2)o1 ; CCOCc1ccc(C)c(Nc2ncc(-c3cc(OC(F)(F)F)cc(N4CCNC4=O)c3)o2)c1 ; COCc1ccc(C)c(Nc2ncc(-c3cc(OC(C)(C)C)cc(N4CCNC4=O)c3)o2)c1]

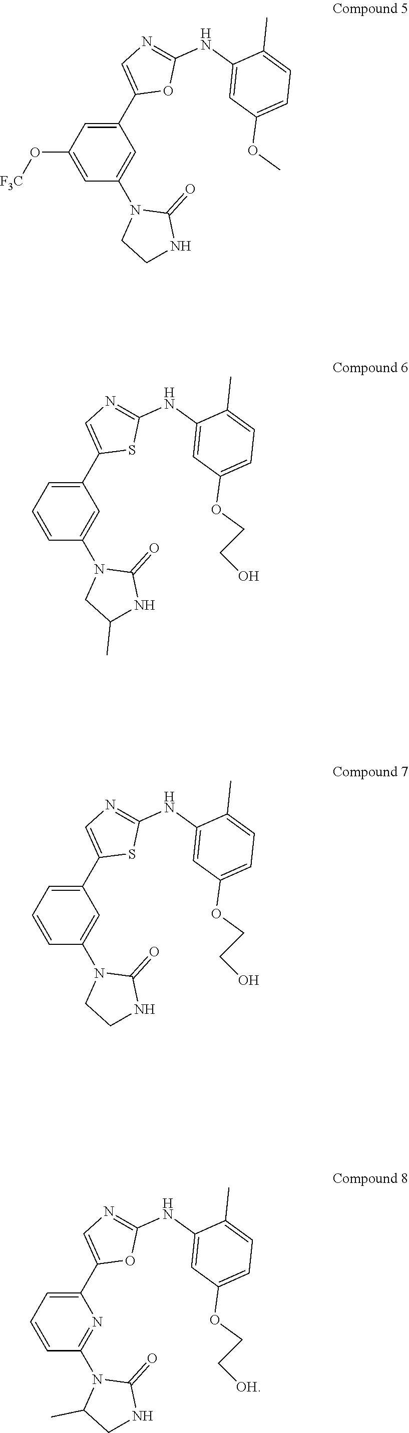 [SMILES: COc1ccc(C)c(Nc2ncc(-c3cc(OC(F)(F)F)cc(N4CCNC4=O)c3)o2)c1 ; Cc1ccc(OCCO)cc1Nc1ncc(-c2cccc(N3CC(C)NC3=O)c2)s1 ; Cc1ccc(OCCO)cc1Nc1ncc(-c2cccc(N3CCNC3=O)c2)s1 ; Cc1ccc(OCCO)cc1Nc1ncc(-c2cccc(N3C(=O)NCC3C)n2)o1]

4. The pharmaceutical composition of claim 1 comprising
cromolyn,
terpene alcohol, and
a pharmaceutically acceptable excipient and/or carrier.

5. The pharmaceutical composition of claim 1 comprising
a tyrosine kinase inhibitor selected from Masitinib and TKI D816V c-kit,
terpene alcohol, and
a pharmaceutically acceptable excipient and/or carrier.

6. The pharmaceutical composition of claim 1, wherein terpene alcohol is selected from the group consisting of cedrenol, cedrol, geraniol, nerolidol, bisabolol, citronellol, nerol, terpineol, linalool, menthol, pulegol, carveol, pinocampheol, myrcenol, isopulegol, famesol, lanceol, santalols, vetiverol, viridiflorol, valerianol, tumerols, patchoulol, occidol, nootkatol, jinkoh eremol, hanamyol, guaicol germacradienol, fokienol, eudesmol, cadinol, an active optical or steric isomer of these compounds, and a combination of one or more thereof.

7. The pharmaceutical composition of claim 1 comprising
cromolyn
bisabolol, and
a pharmaceutically acceptable excipient and/or carrier.

8. The pharmaceutical composition of claim 1, wherein the cromolyn is selected from the group consisting of cromolyn sodium, cromolyn lysinate, and magnesium cromoglycate.

9. A method for treating or lessening the symptoms of, or preventing the symptoms of cutaneous mastocytosis, the method comprising administering the pharmaceutical composition of claim 1 to the subject in need thereof.

10. The method of claim 9, wherein the subject to be treated has a negative D816V c-Kit mutation status and wherein the pharmaceutical composition comprises Masitinib.

11. The method of claim 9, wherein the subject to be treated has a positive D816V c-Kit mutation status and wherein the pharmaceutical composition comprises TKI D816V c-kit.

12. The method of claim 9, wherein the method comprises applying the pharmaceutical composition to the affected skin of the subject once a day.

13. The method of claim 9, wherein the method comprises applying the pharmaceutical composition to the affected skin of the subject multiple times a day.
